(19) United States
(12) Patent Application Publication
(10) Pub. No.: US 2009/0130063 A1
(43) Pub. Date: May 21, 2009

(54) PROCESS FOR SEPARATING AND DETERMINING THE VIRAL LOAD IN A PANCREATIN SAMPLE

(75) Inventors: Dietmar Becher, Diedrichshagen (DE); Leopold Doehner, Greifswald (DE); Frauke Rueffer, Seesen (DE); Martin Frink, Wedemark (DE)

Correspondence Address: MAYER BROWN LLP, P.O. BOX 2828, CHICAGO, IL, 60690, US

(73) Assignee: Solvay Pharmaceuticals GmbH, Hannover (DE)

(21) Appl. No.: 12/271,480

(22) Filed: Nov. 14, 2008

Related U.S. Application Data
(60) Provisional application No. 60/988,323, filed on Nov. 15, 2007.

(51) Int. Cl. A61K 45/00; C12N 9/94; A61P 1/14; C12Q 1/70

(52) U.S. Cl. 424/93.6; 435/186; 435/5

(57) ABSTRACT

Processes for separating a viral load from a pancreatin sample and for quantitatively determining the viral load in a pancreatin sample with high sensitivity are described herein.

### CROSS-REFERENCE TO RELATED APPLICATIONS

This application claims the benefit of U.S. Provisional Application Ser. No. 60/988,323 filed on Nov. 15, 2007 and is hereby incorporated by reference to the extent permitted by law.

### FIELD

Processes for separating an infectious viral load from a pancreatin sample and for quantitatively determining the viral load in a pancreatin sample are described herein.

### BACKGROUND

Pancreatin is a mixture of several physiologically-active constituents and is typically derived from mammalian pancreatic glands. The main constituents of pancreatin are digestive enzymes, in particular pancreatic lipase, amylases and proteases. Due to its important therapeutic properties and high level of safety, pancreatin has long been used as a pharmaceutical preparation in enzyme replacement therapy. Pancreatin has been used to treat pancreatic exocrine insufficiency which is often associated with cystic fibrosis, chronic pancreatitis, post-pancreatectomy, post-gastrointestinal bypass surgery (e.g. Billroth II gastroenterostomy) and ductal obstruction from neoplasm (e.g. of the pancreas or common bile duct). The therapeutic benefits of pancreatin are generally attributed to pancreatic lipase as well as the amylases and proteases. Pancreatin is typically derived from cattle (“bovine pancreatin”) or pigs (“porcine pancreatin”), with porcine pancreatin being more significant in terms of quantity of pancreatin produced. Methods for the production of pancreatin for therapeutic purposes have been previously described, for example in U.S. Pat. No. 4,623,624.

Due to the nature of animal derived pancreatin, the starting materials are typically accompanied by unwanted biological components, such as bacterial or viral contaminants. However, during more than 100 years of commercialization of pharmaceutical products containing pancreatin, no case has been reported where patients have been affected by viral-contaminated pancreatin. Nevertheless, companies producing pharmaceutical products derived from biological tissues and/or body fluids are experiencing additional pressure from the regulatory bodies to increase the level of safety of their products by reducing all contaminants to the lowest level possible, independent of whether any concerned contaminant is considered a human pathogen or not. For the manufacture and use of pharmaceutical products containing pancreatin, it is therefore desirable to have reliable analytical methods for detecting and quantifying such biological contaminants.

To date, no reliable method has been developed for quantitatively detecting or separating viral contaminants in a pancreatin sample. This is likely due to the fact that the enzymatically active constituents of pancreatin are incompatible with the cell lines typically used for multiplying viruses, thus making it more difficult or even impossible to determine the virus titer in a pancreatin sample.

Only recently has a method been developed to allow separation and detection of the viral load in a pancreatin sample, see international patent application PCT/EP2007/054880. However, since the viral load of the preparations derived from animals, like pancreatin, is very low, there is a need for providing a method having high sensitivity for separating and determining the viral load in pharmaceutical preparations, in particular, in pancreatin preparations for pharmaceutical use.

Publication JP 12856990 discloses a method for concentrating or isolating hepatitis viruses by a nonspecific combination of low-speed centrifugation and ultracentrifugation.

### SUMMARY

Described herein is a process for separating the viral load in a pancreatin sample from other components of the sample using a multistage centrifugation process. An additional embodiment is a process for quantitatively determining the viral load of the pancreatin specimen, in particular the infectious viral load of the pancreatin specimen. Another embodiment further includes a quantitative determination of the viral load of the pancreatin specimen which is carried out by determining the virus infection titer in the target fraction containing the viral load and determining the infectious viral load in the test sample. Additional embodiments include methods for treating pancreatin exocrine insufficiency, pharmaceutical compositions comprising pancreatin and processes for the manufacture of pancreatin.

Other embodiments, objects, features and advantages will be set forth in the detailed description of the embodiments that follows, and in part will be apparent from the description, or may be learned by practice, of the claimed subject matter. These objects and advantages will be realized and attained by the processes and compositions particularly pointed out in the written description and claims hereof. The foregoing Summary has been made with the understanding that it is to be considered as a brief and general synopsis of some of the embodiments disclosed herein, is provided solely for the benefit and convenience of the reader, and is not intended to limit in any manner the scope, or range of equivalents, to which the appended claims are lawfully entitled.

### DESCRIPTION

While the claimed subject matter is capable of being embodied in various forms, the description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

The process described herein is suitable for all types of animal-derived pancreatin, such as those of porcine or bovine origin. Additionally, the processes described herein are suitable for separating a viral load from a pancreatin sample to allow for subsequent titer level determination. Viruses which can be quantized according to the methods described herein include bovine rotavirus A, encephalomyocarditis virus (EMCV), porcine circovirus (PCV), porcine parvovirus (PPV), porcine rotavirus A, porcine teschovirus and swine vesicular disease virus (SVDV). Due to their similar properties, the human coxsackievirus B 5/1 may be used as a substitute for SVDV, bovine rotavirus A (for example strain B 223) may be used as a substitute for porcine rotavirus A in assessing the processes described herein. The process described herein is also suitable to determine the viral load of hepatitis E virus (HEV) in a pancreatin specimen by separating the viral load as described and subsequently using further analysis such polymerase chain reaction (PCR) or the like.

One embodiment described herein is a method for separating components of a pancreatin specimen comprising a viral load, the method comprising the steps of:
a) producing a liquid pancreatin test sample suitable for centrifugation from the pancreatin specimen without substantially altering the viral load thereof;
b) subjecting the pancreatin test sample from step a) to centrifugation wherein viruses with sedimentation constants greater than about 120 S do not form a pellet;
c) discarding any solid deposit optionally arising in process step b) during centrifugation and retaining the resulting test sample supernatant;
d) subjecting a portion of the pancreatin test sample supernatant obtained in step c) to a first ultracentrifugation in a discontinuous gradient medium comprising a lowest concentration gradient component and a next higher concentration gradient component, wherein the duration of the first ultracentrifugation and the relative centrifugal force for the first ultracentrifugation are selected such that a portion of the viral load is transported out of the pancreatin test sample supernatant into a first target fraction situated above or in the boundary layer between the lowest concentration gradient component and the next higher concentration gradient component, and removing the first target fraction which (i) contains a portion of the viral load from the pancreatin test sample supernatant; and (ii) may optionally include a pellet present after centrifugation;
e) subjecting the first target fraction obtained in step d) to a second ultracentrifugation wherein the second ultracentrifugation is carried out with a relative centrifugal force higher than the relative centrifugal force of the first ultracentrifugation on a gradient medium wherein the gradient medium is at a higher concentration than the lowest concentration gradient component of the first target fraction; and
f) obtaining a second target fraction containing a portion of the viral load by removing about the upper 75% (vol./vol.) of liquid of an upper layer which corresponds to the first target fraction and obtaining a lower fraction corresponding to (i) about the lower 25% (vol./vol.) liquid of the upper layer; and (ii) the complete layer of the gradient medium of step e) excluding any pellet optionally present after centrifugation.

In process step a) of the embodiment set forth above, a liquid pancreatin test sample suitable for centrifugation is produced from a pancreatin specimen without changing or modifying the viral load, in particular the infectious viral load, thereof. This may, for example, result from producing a pancreatin test sample suspension from the pancreatin specimen. For example, a pancreatin test sample suspension is produced by combining the pancreatin specimen with a cell culture medium (which is suitable for the cell line used to culture the virus species to be investigated) with one or more suitable antibiotic(s). In another embodiment, the pancreatin test sample suspension is produced by combining the pancreatin specimen with a saline solution, in particular, a phosphate-buffered saline solution (PBS at a pH of about 7.2).

In general, any antibiotic is suitable for inclusion in the liquid pancreatin test sample, such as broad-spectrum antibiotics or mixtures of broad-spectrum antibiotics. One or more antibiotics may be selected from the group comprising beta-lactam antibiotics such as penicillins, cephalosporins (including oxacephems and carbacephems), carbapenems and monobactams; streptomycin (including streptomycin sulfate); neomycins (including neomycin A, neomycin B and paromomycin); kanamycins (including kanamycin, gentamicin, amikacin and tobramycin); spectinomycin; tetracyclins (including tetracyclin, oxytetracyclin, doxycyclin and minocyclin); macrolide antibiotics (including erythromycin, clarithromycin, roxithromycin, azithromycin, josamycin and spiramycin); gyrase inhibitors (including nalidixic acid, cinoxacin, pipemidic acid, norfloxacin, pefloxacin, ciprofloxacin, ofloxacin and fleroxacin; folic acid antagonists (including sulfonamide antibiotics, diamino benzylpyrimidines and their combinations); chloramphenicol; lincosamides; glycopeptide antibiotics (including vancomycin and teicoplanin); fosfomycin; polypeptide antibiotics (including polymixin B, colistin, bacitracin and tyrothicin) and mupirocin. Other suitable antibiotics can be found in Remington's: The Science and Practice of Pharmacy, 21^{st} ed., The Merck Index, 14^{th} ed. and Goodman and Gilman's, The Pharmacological Basis of Therapeutics, 11^{th} ed. each of which are hereby incorporated by reference in their entirety. The liquid pancreatin test sample may also include one or more solvents which are compatible with pancreatin and any antibiotic(s) which may be used.

An additional embodiment relates to a process for quantitatively determining the viral load of the pancreatin specimen, in particular the infectious viral load of the pancreatin specimen. For example, one embodiment further comprises process step g) to be carried out at a convenient time during the process (e.g., after process step f), a quantitative determination of the viral load of the pancreatin specimen is carried out by determining the virus infection titer in the target fraction containing the viral load and determining the infectious viral load in the test sample.

The pancreatin test sample suspension is conventionally produced with cooling to a temperature of between about 0° C. and about 15° C., for example to a temperature of between about 4° C. and about 10° C. In a further embodiment the test sample suspension is cooled to about 0° C., about 1° C., about 2° C., about 3° C., about 4° C., about 5° C., about 6° C., about 7° C., about 8° C., about 9° C., about 10° C., about 11° C., about 12° C., about 13° C., about 14° C. or about 15° C.

The constituents of the pancreatin test sample suspension are conventionally stirred in an ice bath for a duration of at least about 30 minutes, for example between about 30 and about 120 minutes, preferably between about 45 and about 90 minutes, in particular about 50 minutes or about 60 minutes. In a further embodiment the constituents of the pancreatin test sample suspension are stirred in an ice bath for a duration of about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes or about 120 minutes. One purpose for cooling the pancreatin test sample suspension and of cooling in other process steps is to avoid, or at least substantially reduce, any unwanted deactivation of the viral load by the enzymatically active constituents of the pancreatin specimen.

The cell culture media to be used as a component of the test sample suspension is determined by the viral species to be separated or quantized. Suitable cell cultures are those in which the viral species to be investigated initiates, if possible, a cytopathic effect (CPE) and are used for culturing and detecting a specific virus species. CPE is a modification of virus-infected cells which is recognizable by light microscopy. If a virus species multiplies in the culture cell without CPE, such multiplication may typically be identified by indirect detection methods known to a person of ordinary skill in the art.

If bovine rotavirus A is to be separated or quantized, fetal monkey kidney cells (MA-104 cells) may be used for culturing of the virus. In this case, Dulbecco's Modified Eagle Medium (Dulbecco medium) is a suitable cell culture medium. If EMCV is to be separated or quantized, porcine kidney cells (PK-15 cells) or embryonic porcine kidney cells (SPEV cells) may be used for culturing the virus. In the case of PK-15 cells, Minimal Essential Medium (MEM) is a suitable cell culture medium. In the case of SPEV cells, Dulbecco medium is suitable as the cell culture medium. If PCV is to be separated or quantized, PK-15 cells may be used for culturing the virus. If PPV is to be separated or quantized, porcine kidney cells (SK-6 cells) may be used for culturing the virus with Dulbecco medium being a suitable cell culture medium. If porcine rotavirus A is to be separated or quantized, MA-104 cells may be used for culturing the virus. If porcine teschovirus is to be separated or quantized, PK-15 cells may be used for culturing the virus. If SVDV is to be separated or quantized, SPEV cells may be used for culturing the virus. A person of ordinary skill in the art would recognize that other cell lines and/or cell culture media, other than those specifically identified, are suitable for culturing the particular viral species to be separated or quantized. Viral species and corresponding cell lines suitable for use may be obtained from sources such as the “American Type Culture Collection”, Manassas, USA (ATCC), the “Deutsche Sammlung von Mikroorganismen und Zellkulturen GmbH”, Braunschweig, Germany (DSMZ), the “Friedrich-Löffler-Institut”, Federal Research Institute for Animal Health, Insel Riems, Germany (FLI) or the “Veterinary Service Division” of the “Department of Agriculture and Rural Development”, Belfast, United Kingdom (DARD).

In process step b), the pancreatin test sample obtained in process step a) may either be used in its entirety, or a portion thereof, may be used. In one embodiment the pancreatin test sample obtained in process step a) is used in its entirety.

The process for separating a viral load from a pancreatin sample further comprises subjecting at least part of the liquid pancreatin test sample to low-speed centrifugation. Low-speed centrifugation includes those conditions under which viruses with sedimentation constants of greater than about 120 S, in particular greater than about 120 S, greater than about 150 S, greater than about 200 S, greater than about 250 S, greater than about 300 S, greater than about 350 S, greater than about 400 S, greater than about 450 S, greater than about 450 S, greater than about 500 S, greater than about 1000 S, greater than about 1500 S, greater than about 2000 S, greater than about 2500 S, greater than about 3000 S, greater than about 3500 S, greater than about 4000 S, greater than about 4500 S and greater than about 5,000 S, do not form a pellet. Typically, viruses with sedimentation constants of greater than about 120 S, in particular viruses with sedimentation constants of greater than about 120 S to about 5,000 S, do not form pellets when the low-speed centrifugations are carried out with a relative centrifugal force of less than about 15,000×g. The relative centrifugal force of the low-speed centrifugation is less than about 15,000×g such as about 14,500×g, about 14,000×g, about 13,500×g, about 13,000×g, about 12,500×g, about 12,000×g, about 11,500×g, about 11,000×g, about 10,500×g, 10,000×g, about 9500×g, about 9000×g, about 8500×g, about 8000×g, about 7500×g, about 7000×g, about 6500×g, about 6000×g, about 5500×g, about 5000×g, about 4500×g, about 4000×g, about 3500×g, about 3000×g, about 2500×g, about 2000×g, about 1500×g, about 1000×g and about 500×g, preferably between about 8,000×g and about 15,000×g, more preferably between about 10,000×g and about 13,500×g (such as about 10,000, about 10,100, about 10,200, about 10,300, about 10,400, about 10,500, about 10,600, about 10,700, about 10,800, about 10,900, about 11,000, about 11,100, about 11,200, about 11,300, about 11,400, about 11,500, about 11,600, about 11,700, about 11,800, about 11,900, about 12,000, about 12,100, about 12,200, about 12,300, about 12,400, about 12,500, about 12,600, about 12,700, about 12,800, about 12,900, about 13,000, about 13,100, about 13,200, about 13,300, about 13,400 and about 13,500) and most preferably about 10,800×g.

The duration of low-speed centrifugation is about 5 minutes, usually between about 5 and about 60 minutes, such as about 5 minutes, about 10 minutes, about 15 minutes, about 20 minutes, about 25 minutes, about 30 minutes, about 35 minutes, about 40 minutes, about 45 minutes, about 50 minutes, about 55 minutes and about 60 minutes. In one embodiment the duration of low-speed centrifugation is between about 10 minutes and about 45 minutes, in another embodiment the duration of low-speed centrifugation is between about 10 minutes and about 30 minutes while in another embodiment the duration is about 15 minutes. In one embodiment, low-speed centrifugation is carried out such that the temperature of the liquid pancreatin test sample is between about 0° C. and about 15° C., such as about 0° C., about 1° C., about 2° C., about 3° C., about 4° C., about 5° C., about 6° C., about 7° C., about 8° C., about 9° C., about 10° C., about 11° C., about 12° C., about 13° C., about 14° C. and about 15° C. In one embodiment the temperature of the liquid pancreatin test sample is between about 4° C. and about 10° C. In a further embodiment the low-speed centrifugation is carried out using a refrigerated centrifuge with cooling such that the temperature of the liquid pancreatin test sample is between about 0° C. and about 15° C., such as about 0° C., about 1° C., about 2° C., about 3° C., about 4° C., about 5° C., about 6° C., about 7° C., about 8° C., about 9° C., about 10° C., about 11° C., about 12° C., about 13° C., about 14° C. and about 15° C.

The low-speed centrifugation set forth in process step b) serves to remove suspension constituents from the pancreatin which may be disruptive to the separation and/or quantitative determination of the viral load of a pancreatin specimen (e.g., various insoluble particles). Following sufficient low-speed centrifugation, the resulting test sample supernatant can be used for further processing. The solid deposits obtained by the low-speed centrifugation set forth in process step b) can be discarded in process step c), while the resulting supernatant can be used for the next process step d). Low-speed centrifugation with subsequent discarding of the solid deposits are repeated until solid deposits are no longer observed to form. Usually, no further repetitions will be necessary after about one to three repetitions of low-speed centrifugation followed by discarding the solid deposits. In one embodiment, the solid deposit as obtained in process step b) can be a sediment or pellet.

In one embodiment, the deposit obtained from the low-speed centrifugation is washed one or more times with a suitable washing fluid before being discarded. A suitable washing fluid is, for example, the pancreatin test sample supernatant resulting from low-speed centrifugation. If a washing fluid other than the pancreatin test sample supernatant is used, the washing fluid is combined with the pancreatin test sample supernatant after washing. It is particularly advantageous to wash the deposit in the above-stated manner prior to further processing if the viral load of the pancreatin sample comprises EMCV.

One embodiment described herein comprises a pancreatin test sample supernatant which may be produced according to process steps a)-c) as described above.

The process of separating a viral load from pancreatin further comprises subjecting at least part of the pancreatin test sample supernatant to ultracentrifugation in a discontinuous gradient medium. The conditions of ultracentrifugation (e.g., duration and magnitude of the relative centrifugal force) are selected such that the transportation of the viral load from the pancreatin test sample supernatant to a target fraction of the discontinuous gradient is substantially complete.

A discontinuous two-phase sucrose gradient is used as the discontinuous gradient medium. The discontinuous gradient medium preferably comprises a gradient prepared from a buffered sucrose solution which in one embodiment is about 50% (wt./vol.) and a buffered sucrose solution which is about 20% (wt./vol.). Neutral buffers (i.e. those which buffer around pH 7) may be used as the buffer for the sucrose solutions. A PBS buffer (phosphate-buffered saline buffer, pH 7.2) is preferred. Sterile gradient media can also be used. This two-phase discontinuous sucrose gradient provides suitable sedimentation and thus separation conditions for the viruses typically associated with animal-derived pancreatin. Discontinuous sucrose gradients, in particular the gradient previous described, exhibit suitable osmotic conditions which will not deactivate the viral load. By using ultracentrifugation (such as that described in process step d)), the transfer of the viral load from the pancreatin test sample supernatant into the discontinuous gradient medium is substantially complete. Substantially complete as used in connection with the ultracentrifugation step means that a viral load previously added to a test sample is so completely recovered in the discontinuous gradient medium that the difference between viral titer level of the added viral load and in that recovered after ultracentrifugation is less than or equal to one half step of the base-ten logarithm of virus titer (0.5 log steps). Use of ultracentrifugation in process step d) allows for the viral load to be transported into a target fraction which is sufficiently far from the pancreatin test sample so as to permit mechanical separation of the target fraction from the pancreatin test sample without any remixing or other disruption of the separated phases occurring.

Creation of the discontinuous two-phase gradient and subsequent ultracentrifugation of process step d) is carried out by introducing a volume of the highest concentration gradient medium into an ultracentrifuge tube over which is layered a volume of the next lower concentration gradient media. This process is repeated as many times as desired to obtain a multi-phase gradient medium with the final (top) layer being a volume of liquid pancreatin test sample suitable for centrifugation from which the viral load is to be separated. In the case of a two-phase gradient medium, the volume of the next lower concentration gradient medium is then immediately overlaid with a volume of a liquid pancreatin test sample (pancreatin test sample volume) which is suitable for centrifugation. In the case of a two-phase gradient medium, this yields a sequence of layers in the ultracentrifuge tube from the top down of a first layer comprising the liquid pancreatin test sample (top), then the volume of the lowest concentration gradient medium (middle; for example a 20% (wt./vol.) buffered sucrose solution) and finally the volume of the highest concentration gradient medium (bottom, covering the base of the ultracentrifuge tube; for example a 50% (wt./vol.) buffered sucrose solution). When overlaying the individual volumes, care must be taken to ensure that no turbulence or intermixing occurs at the respective boundaries.

The target fraction of the multi-phase gradient in process step d) is typically (i) a part of the lowest-concentration gradient medium sufficiently removed from the pancreatin test sample volume; and (ii) the complete volume of the next higher concentration gradient medium. In the case of a two-phase gradient medium the target fraction comprises a part of the lowest concentration gradient medium sufficiently far away and remote from the pancreatin test sample volume and the complete volume of the highest concentration gradient medium extending downward to the bottom of the ultracentrifuge tube and optionally including any pellet(s) which have formed at the bottom of the ultracentrifuge tube.

When calculating the location of a target fraction which is sufficiently removed from the pancreatin test sample volume thereby allowing for subsequent separation, it should be borne in mind that the calculated values for the position of the particles (i.e. the position of the viral particles separated from the pancreatin sample) usually denote the vertices of a Gaussian distribution. As such, the particles will be distributed both above and below their calculated position. It is thus necessary when determining the desired distance of the target fraction from the pancreatin test sample volume to include an additional margin to account for the Gaussian distribution of the particle locations. The viral load is transported into a target fraction which is sufficiently distant from the pancreatin test sample volume for subsequent separation if particles with a sedimentation constant of greater than about 120 S, in particular greater than about 120 S to about 5,000 S, have migrated from the pancreatin test sample volume at least about 10 mm, at least about 15 mm, at least about 20 mm, at least about 25 mm or at least about 30 mm, into the lowest concentration gradient medium due to the ultracentrifugation. In the case of the two-phase gradient medium embodiment previously described, the lowest concentration gradient medium is the 20% (wt./vol.) buffered sucrose solution. In one embodiment, substantially all of the viral particles with a sedimentation constant of greater than about 120 S, in particular greater than about 120 S to about 5,000 S, have passed through the lowest concentration gradient medium and are located at the boundary with the next higher concentration gradient medium (i.e. on a “sucrose cushion”). A person of ordinary skill in the art is aware of suitable ways of calculating and implementing the necessary conditions for obtaining the desired results from the ultracentrifugation. Suitable ultracentrifugation conditions may be determined based upon the characteristics of the virus(es) to be separated from the pancreatin sample (e.g., density and sedimentation constant) by a person of ordinary skill in the art (see for example Lebowitz et al., “Modern analytical ultracentrifugation in protein science: A tutorial review”; Protein Science 11 (2002) 2067-79).

Provided that ultracentrifugation is carried out under suitable conditions and volume ratios and in the discontinuous gradient medium, the viral load will be transported into a target fraction of the discontinuous gradient medium and will be available for subsequent separation by the second ultracentrifugation step. Suitable ultracentrifugation conditions include carrying out the ultracentrifugation step for a duration of more than about 1 hour. In further embodiments the ultracentrifugation step is carried out for more than about 4 hours, more than about 9 hours, between about 9 hours and about 20 hours or between about 12 hours and about 18 hours. In other embodiments the ultracentrifugation step is carried out for more than about 1 hour, more than about 2 hours, more than about 3 hours, more than about 4 hours, more than about 5 hours, more than about 6 hours, more than about 7 hours, more than about 8 hours, more than about 9 hours, more than about 10 hours, more than about 11 hours, more than about 12 hours, more than about 13 hours, more than about 14 hours, more than about 15 hours, more than about 16 hours, more than about 17 hours, more than about 18 hours, more than about 19 hours or more than about 20 hours.

A suitable relative centrifugal force for the ultracentrifugation is between about 50,000×g and about 150,000×g, such as about 50,000×g, about 55,000×g, about 60,000×g, about 65,000×g, about 70,000×g, about 75,000×g, about 80,000×g, about 85,000×g, about 90,000×g, about 95,000×g, about 100,000×g, about 105,000×g, about 110,000×g, about 115,000×g, about 120,000×g, about 125,000×g, about 130,000×g, about 135,000×g, about 140,000×g, about 145,000×g or about 150,000×g. In one embodiment the first ultracentrifugation step is carried out for a duration of between about 12 hours and about 18 hours with a relative centrifugal force of between about 50,000×g and about 150,000×g with volume ratios suitable for carrying out ultracentrifugation and in a gradient prepared from a 50% (wt./vol.) buffered sucrose solution and a 20% (wt./vol.) buffered sucrose solution. In a further embodiment the first ultracentrifugation step is carried out for a duration of between about 15 hours and about 17 hours with a relative centrifugal force of between about 70,000×g and about 120,000×g with volume ratios suitable for carrying out ultracentrifugation and in a gradient prepared from a 50% (wt./vol.) buffered sucrose solution and a 20% (wt./vol.) buffered sucrose solution.

Suitable volume ratios for carrying out ultracentrifugation can be obtained, for example, by using conventional ultracentrifuge tubes. Conventional ultracentrifuge tubes are, for example, those having a volume of between about 10 ml and about 50 ml, such as those having a volume of between about 25 ml and about 50 ml. Provided that a conventional ultracentrifuge tube is used in process step d), the volume of the highest concentration gradient medium (for example a 50% (wt./vol.) buffered sucrose solution) may be present in an amount of between about 0.5 ml and about 5 ml, such as about 2 ml. The volume of the next lower concentration gradient medium (for example a 20% (wt./vol.) buffered sucrose solution) may be present in an amount of between about 3 ml and about 20 ml, such as about 14 ml, and the pancreatin test sample volume may be present in an amount of between about 6.5 ml and about 25 ml, such as about 17 ml.

The second ultracentrifugation is carried out in step e) uses the first target fraction obtained in step d) by placing it on a gradient medium cushion. The gradient medium cushion can be the same or different from that used in the first ultracentrifugation where the gradient medium is at higher concentration than the concentration of the lowest concentration gradient component of the first target fraction. For example, if the first ultracentrifugation is performed with a discontinuous gradient centrifugation using the above described system of a 20% wt./vol. buffered sucrose solution and a 50% wt./vol. buffered sucrose solution, the resulting first target fraction would be a system containing a buffered sucrose solution of a concentration of higher than about 20% wt./vol. and below about 50% wt./vol. For purposes of carrying out ultracentrifugation step e), the sucrose cushion may be about a 50% wt./vol. buffered sucrose solution. The first target fraction used in step e) also comprises any pellet optionally present after the first centrifugation.

The second ultracentrifugation is carried out for a duration of more than about 1 hour. In further embodiments the ultracentrifugation step is carried out for more than about 2 hours, between about 2 hours and about 8 hours or between about 3 hours and about 6 hours. In other embodiments the ultracentrifugation step is carried out for more than about 1 hour, more than about 2 hours, more than about 3 hours, more than about 4 hours, more than about 5 hours, more than about 6 hours, more than about 7 hours or more than about 8 hours.

A suitable relative centrifugal force for the second ultracentrifugation step is greater than about 100,000×g such as between about 100,000×g and about 350,000×g or between about 200,000×g and about 350,000×g. In a further embodiment, the relative centrifugal force for the second ultracentrifugation step is about 100,000×g, about 110,000×g, about 120,000×g, about 130,000×g, about 140,000×g, about 150,000×g, about 160,000×g, about 170,000×g, about 180,000×g, about 190,000×g, about 200,000×g, about 210,000×g, about 220,000×g, about 230,000×g, about 240,000×g, about 250,000×g, about 260,000×g, about 270,000×g, about 280,000×g, about 290,000×g, about 300,000×g, about 310,000×g, about 320,000×g, about 330,000×g, about 340,000×g or about 350,000×g. In yet a further embodiment, the relative centrifugal force for the second ultracentrifugation step is between about 200,000×g and about 350,000×g and is carried out for a duration of between about 3 hours and about 6 hours in volume ratios suitable for carrying out the ultracentrifugation. In yet a further embodiment the relative centrifugal force for the second ultracentrifugation step is between about 250,000×g and about 300,000×g and is carried out for a duration of between about 4.5 hours and about 5.5 hours in volume ratios suitable for carrying out the ultracentrifugation.

Suitable volume ratios for carrying out ultracentrifugation can be obtained, for example, by using conventional ultracentrifuge tubes. Conventional ultracentrifuge tubes are, for example, those having a volume of between about 5 ml and about 25 ml, such as those having a volume of between about 10 ml and about 15 ml or having a volume between about 12 ml and about 13 ml, an internal radius of between about 6 mm and about 8 mm and a height of between about 80 mm and about 100 mm, such as between about 85 mm and about 95 mm. Provided that a conventional ultracentrifuge tube is used in process step e), the volume of the gradient cushion (for example a 50% (wt./vol.) buffered sucrose solution) may amount, for example, to about 0.5 ml, and the first target fraction volume may amount, for example, to about 10 ml.

The various processes described herein reduces the limit of detection down to a value of approximately one infectious unit per gram pancreatin specimen used as a starting material.

In one embodiment, independent of the other conditions, the temperature during the ultracentrifugation of steps d) or e) of the pancreatin test sample supernatant is between about 0° C. and about 15° C., for example about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., or about 15° C. and preferably between about 4° C. and about 10° C.

Refrigerated centrifuges which are suitable for use in the processes described herein are known to a person of ordinary skill in the art. Commercially available ultracentrifuges are suitable for use and include refrigerated ultracentrifuges with a swinging-bucket rotor, for example, an ultracentrifuge from Sorvall® with a model TH-641 swinging-bucket rotor.

The foregoing description of the low-speed centrifugation and ultracentrifugation may be scaled up or down to any desired extent and the descriptions provided herein are simply meant to illustrate a few embodiments of the claimed subject matter.

The process described herein for separating a viral load from pancreatin further comprises quantitatively separating the target fraction containing the viral load from the pancreatin test sample supernatant. Separation can be undertaken by placing a mark on the ultracentrifuge tube at the location of the previously determined boundary of the target fraction. The entire volume above this boundary is then separated from the remaining volume by, for example, aspiration. Aspiration may proceed with a peristaltic pump which has been previously sterilized. A suitable pumping rate is about 2 ml/minute. During aspiration, care should be taken to ensure that the peristaltic pump capillary is located at the upper border of the liquid. The target fraction remaining in the ultracentrifuge tube may then be removed with a conventional single-channel pipette, preferably with a sterile tip. Any sediment remaining in the ultracentrifuge tube may be removed at the same time by being repeatedly drawn up in the single-channel pipette and resuspended.

The target fraction containing the viral load separated from the pancreatin test sample supernatant forms an additional embodiment herein.

The process described herein optionally comprises an additional step of quantitatively determining the viral load of the pancreatin sample. In this optional step, the viral load of the pancreatin sample is quantized by determining the virus infection titer in the target fraction containing the viral load. This quantitative determination may proceed in accordance with methods known to those of ordinary skill in the art, for example, in accordance with the principle of virus infection titer determination (VITD).

The second target fraction may, for example, be diluted in a suitable ratio with a compatible cell culture medium in order to obtain a suitable test sample for analysis. Compatible cell culture media include those previously identified in connection with each particular virus under investigation as well as other suitable cell cultures media known to a person of ordinary skill in the art. A further embodiment includes ruling out false positives for viral infections by filtering the diluted or undiluted target fraction through a microfilter before the quantitative determination of the viral load is carried out.

A suitable dilution may, for example, be achieved by diluting the target fraction with the cell culture medium to the original volume of the pancreatin test sample supernatant. Then a series of dilutions of the virus determination test samples may be produced, for example, in dilution steps of 1:2, 1:5 or 1:10 or also in combinations of these dilution steps, in order to carry out a quantitative VITD. Then a cell suspension may be inoculated with the virus determination test samples of different concentrations from the dilution series, whereupon a cell layer is allowed to form on the differing concentrations of the virus determination test samples. To rule out false positives for virus infection which may be caused by the presence of microbials such as inert bacteria or mycoplasms, the virus determination test samples are filtered before inoculating them onto detector cells. A virus determination test sample or a diluted virus determination test sample may be filtered through a microfilter having a pore size (i.e., pore diameter) between about 0.1 and about 10 μm, preferably a microfilter having a pore size (i.e., pore diameter) of between about 0.1 μm and about 10 μm such as about 0.1 μam. The filtrate, which forms yet another embodiment described and claimed herein, may then be used as a test sample for further investigations. The inoculated test samples are then evaluated for their degree of infection depending upon the manner in which their infection is indicated. When CPE is used as an indicator of infection of a cell layer, it is read after between about 4 days and about 7 days. Titration (end-point dilution) of the virus determination test samples permits a quantitative determination of the viral load. Titration conventionally proceeds by dilution by a factor of 10, i.e. based on the base-ten logarithm. In practice, the 50% infection dose (ID₅₀) is usually calculated. In the case of parallel multiple batches, the identified ID₅₀ value then corresponds to that of the highest (reciprocal) dilution of the virus determination test sample at which a CPE is detectable in exactly half the batches. Optionally, the results may also be computationally corrected or interpolated. The most commonly used methods for virus titer calculation are those according to Spearman and Kärber (see C. Spearman, Br. J. Psychol. 2 (1908) 227-242 and G. Kärber, Arch. Exp. Path. Pharmak. 162 (1931) 480-483; also Bundesanzeiger [Federal gazette] no. 84, May 4, 1994) or according to Reed and Muench (see Reed, L. J., Muench, H. Am. J. Hyg. 27 (1938) 493-497).

Other indicators of an infection of the cell layer may also be used, such as virus antigen induction or plaque induction. The person of ordinary skill in the art is familiar with these methods as well as others and the details of their application to the subject matter described herein. Additional information can be found, for example, in virology textbooks such as “Medizinische Virologie” by H. W. Doerr and W. H. Gerlich, Georg Thieme Verlag Stuttgart, New York, 1st edition 2002 or in each case the most recent edition thereof.

The methods described herein are also suitable for analyzing larger quantities of pancreatin as the starting material. Using large amounts pancreatin usually allows one to determine the viral load with a higher sensitivity, namely, to determine lower infectious units per gram. Thus, the methods described herein are suitable for quantities of starting material of greater than about 5 g, such as, for example, about 6 g, about 7 g, about 8 g, about 9 g, about 10 g, about 11 g or about 12 g.

### EXAMPLES

All tasks stated in the following Examples were carried out under sterile conditions on a sterile workbench. The following materials inter alia were used:
1. Antibiotic solution, 1.0 g of streptomycin sulfate and 1.2 g of penicillin were dissolved in 20 ml of twice-distilled water and filtered through a 0.2 μm filter. The filtrates were divided into 1 ml aliquots and optionally stored at −20° C. until use;
2. Dulbecco medium, cell culture medium for SK 6 cells, SPEV cells and MA 104 cells;
3. Single channel pipette, with sterile tips;
4. FCS, fetal calf serum (serum).
5. Tissue culture flasks, sterile, area of flask base in each case 25, 75 or 175 cm²;
6. MEM, cell culture medium for PK-15 cells with 1.5 g/l sodium bicarbonate and 1 mM pyruvate;
7. Microtiter plates, sterile with 96 wells and lid;
8. PAN suspension, 10% pancreatin suspension; 8.0 g of porcine pancreatin (unless otherwise stated) weighed out under sterile conditions into a beaker, combined with 8 ml of antibiotic solution and (unless otherwise stated) 64.0 ml of the particular corresponding cell culture medium or PBS and (unless otherwise stated) suspended within 60 minutes in an ice bath with stirring;
9. Pardee buffer, Pardee's carbon dioxide buffer;
10. PBS, sterile “phosphate buffered saline” solution (pH 7.2);
11. Pipettes, sterile;
12. Pipette tips, sterile in sterile trays;
13. Plastics pouches, CO₂-impermeable with closure (“Anaerocult®”, from Merck);
14. Polyclonal anti-PPV antibody, fluorescein isothiocyanate (FITC) conjugate, from NatuTec GmbH;
15. Tubes, sterile 15 and 50 ml;
16. Sucrose solution, 20%, PBS-buffered, sterile; concentration is adjusted with the assistance of a refractometer;
17. Sucrose solutions, 50%, PBS-buffered, sterile; concentration is adjusted with the assistance of a refractometer;
18. Screw-top tubes, sterile;
19. Trypsin solution, “TrypL Express®”, from INVITROGEN;
20. Peristaltic pump, from “ismaTec”, pumping rate up to 5.8 ml/minute;
21. Refrigerated ultracentrifuge, refrigerated centrifuge BIOFUGE® 22R Heraeus SEPATECH® with fixed angle rotor No. 3745;
22. Ultracentrifuge tubes, sterile, capacity 11 ml, dimensions 9×90 mm;
23. Dilution blocks, 96 wells each of 1.0 ml;
24. MA-104 cells: supplied by FLI;
25. PK-15 cells: supplied by DARD;
26. SK-6 cells: supplied by FLI;
27. SPEV cells: supplied by FLI;
28. Cell suspensions of the SK 6, SPEV and PK-15 cells to be tested with 200,000 cells/ml in cell culture medium with 10% FCS;
29. Sterile Falcon microtubes, capacity 15 ml;
30. determination (VITD)

### Example 1

### Investigation of the Negative Effect of Pancreatin on Various Cell Lines

For the purposes of detecting viruses in material test samples using cell cultures, the negative effect of the pancreatin sample under investigation on the cells should be ascertained so that false negative results can be eliminated when evaluating CPE. As stated below, investigations to ascertain the negative effect of a pancreatin test sample suspension were carried out on various cell lines.

0.5 ml portions of a PAN suspension produced as described above were taken in order to evaluate any negative effect. These 0.5 ml portions were designated “pancreatin suspension test sample”.

Low-speed centrifugation: The remaining PAN suspension was centrifuged for 15 minutes at 10,000 rpm (10,800×g) and 4° C. in a conventional refrigerated centrifuge (Megafuge® 1.0R Heraeus SEPATECH® with swinging-bucket rotor no. 2704). After low-speed centrifugation, the supernatant was then centrifuged for a further 15 minutes at 10,000 rpm and 4° C. and designated “supernatant after low-speed centrifugation” to be used for virus titration and ultracentrifugation. The two sediments resulting from both low-speed centrifugations were combined (together 1 ml), resuspended in 9 ml of the respective cell culture medium and designated “sediment”.

The first ultracentrifugation: A test sample was taken from the “supernatant after low-speed centrifugation” and subjected to a first ultracentrifugation in an ultracentrifuge. In preparation for ultracentrifugation, 2 ml of 50% (wt./vol.) sucrose solution was introduced with a pipette into the number of ultracentrifuge tubes necessary for carrying out testing. With the ultracentrifuge tube held at an oblique angle, 14 ml of a 20% (wt./vol.) sucrose solution was carefully placed on top of the 50% (wt./vol.) sucrose layer with a dividing layer being discernible between the two solutions. A 17 ml layer of the “supernatant after low-speed centrifugation” was then carefully placed on top of the 20% (wt./vol.) sucrose solution to avoid turbulence and intermixing. The ultracentrifuge tubes were then placed in the ultracentrifuge rotor. The two ultracentrifuge tubes were placed on opposite sides of the rotor and counterbalanced with ultracentrifugation tubes containing PBS, inserted into the corresponding holders and tightly sealed with their respective lids. Upon insertion into the rotor, the test samples were centrifuged for 16 hours at 10° C. and 22,000 rpm (87,000×g). After ultracentrifugation, the ultracentrifuge tubes were removed from the holders on a sterile workbench and marked at a height of 5 cm from the bottom of the ultracentrifuge tube. Using a peristaltic pump with sterilized tubing and capillaries, the liquid above the mark was aspirated from the ultracentrifuge tube at a pumping rate of 2 ml/minute with the capillary being located at the upper border of the liquid. The first fraction obtained in this manner was designated “upper fraction after first ultracentrifugation.” The “lower fraction after first ultracentrifugation” (1.5 ml) remaining in the ultracentrifuge tube was removed from the ultracentrifuge tube with the assistance of a single-channel pipette. Any sediment possibly remaining on the bottom of the ultracentrifuge tube was resuspended by being repeatedly drawn up with the single-channel pipette and likewise removed. The “lower fraction after first ultracentrifugation” from all tubes was combined and used in the second ultracentrifugation. The resultant fractions were stored at 4° C. until further processing or stored at −20° C. in the case of extended storage.

The second centrifugation step was carried out in two 11 ml-tubes for 5 hours at 4° C. and at 272,000×g. A gradient medium cushion of 50% (wt./vol.) sucrose solution was first introduced into the tubes (0.5 ml). Five milliliters of the first target fraction were then layered onto the gradient cushion and ultracentrifugation was performed as previously described. After discarding the upper fraction, a volume of 1.5 ml was removed from the bottom of each tube excluding the pellet.

The samples labeled “pancreatin suspension test sample”, “supernatant after low-speed centrifugation”, “sediment” (after low-speed centrifugation), “upper fraction after ultracentrifugation” and “lower fraction after ultracentrifugation” (after being made up to 5.0 ml) were then tested for any negative effect on various cell lines. To achieve this, a series of dilutions of the test samples were produced. All the test samples were further diluted by a factor of two from a dilution of 1:5 with the respective cell culture medium. In eight parallel microtiter plates, 100 μl portions of cell suspension comprising PK-15, SPEV or SK 6 cells were added to each well. Also added to each well was 100 μl of the test sample dilutions. When MA 104 cells were tested, microtiter plates with a 24-hour old cell layer were used. The cell culture medium was removed from the wells and replaced with 100 μl of fresh cell culture medium without serum to yield the final test sample dilutions of 1:10, 1:20, 1:40, 1:80, 1:160 etc. As a control, 100 μl of cell culture medium were introduced into eight wells of each microtiter plate instead of 100 μl from the dilution series. Pairs of plates together with a tube containing 4 ml of Pardee buffer and filter paper were placed in air-tight pouches and tightly sealed. The plates were then incubated at 36±1° C. for up to 7 days. Over the period of incubation, the plates were inspected daily by microscope for the extent of CPE, i.e. for cell lysis and/or degeneration of the cells and for the absence of the formation of a cell layer as a result of the negative effect of the pancreatin. The final evaluation was carried out after seven days. The titration was repeated if cell degeneration had already occurred in the controls on the final reading.

The results of testing the different samples demonstrated that there was no negative effect on the various cell lines of the “lower fraction after second ultracentrifugation” derived from pancreatin.

The results demonstrated that in the “lower fraction after first ultracentrifugation” and “the lower fraction after the second ultracentrifugation” (which has been subjected to the first or first and second ultracentrifugation step as previously described and in which the viral load has been concentrated) there is a considerable reduction in the negative effect towards the investigated cell lines relative to the other test samples investigated.

### Example 2

### Testing of Different Pancreatin Charges on Viral Load

The two step ultracentrifugation procedure described herein was used to isolate viruses from suspensions of different charges of pancreatin powder. The separation of the viruses from the pancreatin was used for the detection of low virus loads by titration on susceptible cell lines:

8 g of drug substance were mixed with 8 ml of antibiotic solution (1.0 g streptomycin sulfate and 1.2 g Penicillin G per 20 ml of sterile PBS) and 64 ml of PBS and stirred in an ice water bath for 60 minutes

the resulting suspension was centrifuged at 4° C. for 15 minutes at 10,800×g

the supernatant after first centrifugation was centrifuged again at 4° C. for 15 minutes at 10,800×g.

The resulting supernatant after low-speed centrifugation was used for a first ultra centrifugation. The first ultra centrifugation was performed with a discontinuous sucrose gradient (four centrifugation tubes with 2 ml of 50% (wt./vol.) sucrose and 14 ml of 20% (wt./vol.) sucrose were covered with 17 ml of supernatant after low speed centrifugation). This technique allows for the separation of virus particles from the main part of the pancreatin suspension. The viruses from virus/pancreatin suspension mixtures become concentrated in a border layer between the 50% (wt./vol.) and 20% (wt./vol.) sucrose layers under appropriate centrifugation conditions. The soluble pancreatin components remain in the supernatant after centrifugation above the virus-containing layer. The virus fraction is separated from the pancreatin layers by fractionated removal (e.g., suction) of the sucrose gradient. The resulting virus fraction was ultra centrifuged again for further concentration of viruses on a 50% (wt./vol.) sucrose cushion.

The first centrifugation was carried out in four 36 ml-tubes for 16 hours at 4° C. and at 87,000×g. After centrifugation, a volume of 5 ml was removed from the bottom of each tube.

The second centrifugation was subsequently carried out in two 11 ml-tubes for 5 hours at 4° C. and at 272,000×g. A volume of 1.5 ml was removed from the bottom of each tube. This resulted in a 3 ml virus concentrate which was obtained from 8 g of pancreatin powder. The samples were stored at −20° C.

Infectivity Assays

The infectivity assay is based on the assumption that a low virus load of a sample can be amplified to detectable levels by passing the sample through susceptible cell lines three consecutive times. The viruses which are present can infect new cells and become amplified during this process leading to an increased virus titer. In combination with the large volume plating technique, it is possible to detect even a single infectious virus with a low amplification rate of 10 (10 viruses per 1 infectious virus within 6 days of incubation) during three cell passes by the formation of about 1000 plaques on a cell lawn after the third pass.

The virus fractions extracted from the pancreatin samples were applied to three consecutive passes on SK 6 cells for detection of porcine Parvoviruses.

First Pass (1 ml Virus Fraction=2.66 g Pancreatin)

One-third of each individual virus concentrate (1 ml of 3 ml) was inoculated in a 75 cm² tissue culture flask containing 30 ml cell culture medium with 5% of FCS and a 24 h old subconfluent cell lawn of SK 6 cells. The cell culture flasks were incubated at 37±1° C. for 6 days.

The formation of cytopathic effects (plaques) was checked with an inverse microscope during, and at the conclusion of, the incubation period. The first pass was completed with the creation of a virus/cell lysate by two freeze thaw cycles of the total tissue and culture content of the flask. The virus cell lysate was subjected to centrifugation (2,800×g for 15 minutes) to create a cell-free supernatant. This supernatant resulting after centrifugation (30 ml per lot) was filtered through a filter with 0.1 μm pore size as a safety step to reduce a possible contamination of the lysate with mycoplasmas.

Second Pass

A 75 cm² tissue culture flask containing 22.5 ml cell culture medium with 5% FCS and a 24 h old cell lawn of SK 6 cells was prepared for the second pass of each lot. 7.5 ml of cell culture supernatant from the first pass (25%) was added. Flasks were incubated for 6 days at 37±1° C. and were checked for cytopathic effects with the aid of an inverse microscope.

The virus/cell culture lysate (30 ml per lot) was achieved as described for the first pass.

Third Pass

A 75 cm² tissue culture flask was prepared for each lot as described above. 7.5 ml of the virus/cell culture lysate from the second pass (25%) and 22.5 ml cell culture medium with 5% FCS were added per flask. Flasks were incubated for 6 days at 37±1° C. and inspected for cytopathic effects with the aid of an inverse microscope.

The virus/cell culture lysate (30 ml per lot) was achieved as described for the first and second passes.

4. Detection of Porcine Parvovirus (PPV) by Immuno-Staining with FITC-Conjugated Anti-PPV Antibodies

Micro titer plates with 96 wells and a 24 hour old cell lawn of SK 6 cells were prepared. Thirty cavities were infected with 100 μl each of the virus/cell culture lysate from the third pass (10%) and incubated at 37±1° C. for 24 hours.

After incubation the cell culture supernatant was removed from the cells and the cell lawn was fixed by an ice cold mixture of 80% acetone and 20% methanol. 100 μl of FITC-conjugated anti-PPV antibody solution per cavity were added and the plates were incubated for 45 minutes at 37±1° C. After removing the antibody solution the cell lawn was washed three times with PBS and covered with 15% glycerol in PBS. The detection of cells infected by Parvovirus was carried out with the aid of an inverse microscope in UV-light.

In parallel with the pancreatin samples, porcine Parvovirus NADL-2 with known titer was titrated on the same microtiter plates as positive control.

All test items were passed on SK 6 cells in parallel with the positive controls as described. In this manner, it was possible to compare the formation of plaques/cytopathic effects typical for porcine Parvovirus between pancreatin samples and positive controls.

The results of cultivation are shown in Table 1.

TABLE 1
Results of passing the test items on SK 6 cells in cell tissue culture flasks

| Lot number of | Passing of virus fractions of pancreatin samples on SK 6 cells in tissue | | |
|---|---|---|---|
| pancreatin | culture flasks with inspection for cytopathic effects | | |
| powder | First Pass | Second Pass | Third Pass |
| 0436 | no CPE detected | no CPE detected | no typical CPE |
| | | | detected but slight cell |
| | | | degeneration 6 days |
| | | | post infection |
| 0437 | no CPE detected | single plaques in cell | 30% of cell lawn with |
| | | lawn 4 days post | plaques 6 days post |
| | | infection, but non- | infection |
| | | confluent CPE 6 days | |
| | | post infection | |
| 0438 | no CPE detected | 4 days post infection | 2 days post infection |
| | | 80% of cell lawn with | visible plaque |
| | | plaques, confluent | formation; confluent |
| | | CPE 6 days post | CPE 4 days post |
| | | infection | infection |

After the first pass of the three test items no CPE was detected 6 days post infection.

No CPE was detectable during three passes of sample 0436. Only in the third pass of sample 0436 was a slight cell degeneration observed. This was, however, not identical to the observed cytopathic effects of samples 0437 and 0438.

Single plaques in the cell lawn were observed during the second pass of sample 0437 four days post infection. At 6 days post infection approximately 20% of the cell lawn in the flask showed CPE. After the third pass of the sample about 30% of the cell lawn showed cytopathic effects/plaques.

Single plaques were observed 3 days post infection during the second pass of sample 0438. Further passing of the plaque formation continued and led to nearly confluent CPE.

In the third pass a clear CPE was detected 2 days post infection. The incubation was finished 4 days post infection with a confluent CPE.

Subsequent to the third pass in tissue culture flasks, the virus/cell culture lysate from the third pass was transferred to micro titre plates with SK 6 cell lawn:

30 times 100 μl per lot into 30 wells in two parallels. A positive control of porcine Parvovirus NADL-2 was titrated on the same plates.

Plates were incubated at 37±1° C. with the first plate used for observing cytopathic effects while the second plate was used for detection of porcine Parvovirus by immunostaining.

The first plate was incubated for 6 days. At the end of incubation, samples 0437 and 0438 showed the same cytopathic effect as the positive control PPV.

NADL-2. The sample 0436 did not show any CPE.

The culture supernatant from the second micro titre plate was removed 24 hours after starting the incubation. The short incubation time should reduce the detectability of cells with secondary infections by parvoviruses which were released from primary infected cells. Cell lawn was fixed and stained with an FITC-conjugated anti-PPV-antibody and checked for infected cells with the aid of an inverse microscope in UV-light. This corresponds with a plaque titration and allows for possible detection of the virus titre of the third pass lysate.

The following results were obtained:

None of the 30 cavities which were infected with the cell-free virus lysate from the third pass of sample 0436 contained distinct fluorescent cells.

In addition to the 3 ml (10%) that were tested, a further 30×100 μl of cell-free virus lysate were transferred into micro titre plate with SK 6 cell lawn, incubated for 24 hours at 37±1° C., fixed and stained with anti-PPV antibody. Non-infected cells were found in these 30 additional cavities.

This result correlates with the results of passing the sample 0436 in tissue culture flasks and after a third pass in micro titre plates for detection of cytopathic effects.

In the case of samples 0437 and 0438, all 30 cavities which were stained with anti-PCV antibody contained distinct fluorescent cells such as the positive control PPV NADL-2.

The process described above allows one to identify one infectious unit per 1 g pancreatin, and, thus, provides a highly sensitive process for separating and determining viral load from a pancreatin specimen. Further, in contrast to processes based on molecular biology methods like detection of nucleic acids molecules based on amplification methods like PCR, the process described herein allows for determining infectious units. It is therefore possible to differentiate between infectious and non-infectious viral-load or total viral load determined by molecular biology techniques known to a person of ordinary skill in the art.

In a further embodiment, the passages and the detection can be performed sequentially (as described above) or in parallel. In parallel means, that while one passage is being performed the virus load of the previous passage can be determined. Using this approach, a result can be obtained from all passages and if the result is obtained from the first passage while a result is achieved from an earlier point of time as using the stepwise approach. This can be of importance, for example, to increase time efficiency and/or, for example, for the quality assurance in manufacturing processes as the respective sample or product can be released at an earlier point in time.

Pharmaceutical Compositions

Another embodiment described herein includes a process for manufacturing a pharmaceutical composition comprising pancreatin wherein such pharmaceutical composition can be in a dosage form suitable for oral administration. The oral dosage form can be for immediate and/or modified release, such dosage form can be tablets, microtablets, pellets, micropellets, microspheres, granules, granulates, powders, suspensions, emulsions, dispersions, capsules, sachets as well as other suitable dosage forms.

In one embodiment the pharmaceutical composition comprises pancreatin which is optionally coated with one or more gastric-acid resistant coatings known to a person of ordinary skill in the art.

In another embodiment of the pharmaceutical composition, the optionally gastric-acid resistant coated pancreatin or its dosage form is further filled into sachets and/or capsules.

A further embodiment described herein are pharmaceutical compositions which can be prepared utilizing the processes described herein. The pharmaceutical compositions can comprise
a) a pharmacologically effective quantity of pancreatin having a viral load, wherein the viral load of the pancreatin has been quantitatively separated by the process comprising the steps of:
i) producing a liquid pancreatin test sample suitable for centrifugation from the pancreatin specimen without substantially altering the viral load thereof;
ii) subjecting the pancreatin test sample from step a) to centrifugation wherein viruses with sedimentation constants greater than about 120 S do not form a pellet;
iii) discarding any solid deposit optionally arising in process step b) during centrifugation and retaining the resulting test sample supernatant;
iv) subjecting a portion of the pancreatin test sample supernatant obtained in step c) to a first ultracentrifugation in a discontinuous gradient medium comprising a lowest concentration gradient component and a next higher concentration gradient component, wherein the duration of the first ultracentrifugation and the relative centrifugal force for the first ultracentrifugation are selected such that a portion of the viral load is transported out of the pancreatin test sample supernatant into a first target fraction situated above or in the boundary layer between the lowest concentration gradient component and the next higher concentration gradient component, and removing the first target fraction which (i) contains a portion of the viral load from the pancreatin test sample supernatant; and (ii) may optionally include a pellet present after centrifugation;
v) subjecting the first target fraction obtained in step d) to a second ultracentrifugation wherein the second ultracentrifugation is carried out with a relative centrifugal force higher than the relative centrifugal force of the first ultracentrifugation on a gradient medium wherein the gradient medium is at a higher concentration than the lowest concentration gradient component of the first target fraction; and
vi) obtaining a second target fraction containing a portion of the viral load by removing about the upper 75% of liquid of an upper layer which corresponds to the first target fraction and obtaining a lower fraction corresponding to (i) about the lower 25% liquid of the upper layer; and (ii) the complete layer of the gradient medium of step e) excluding any pellet optionally present after centrifugation; and
b) one or more pharmaceutically acceptable excipients.

The viral load can optionally be reduced as part of manufacturing a pharmaceutical composition as described herein. Methods of reducing viral loads in pancreatin are known to those of ordinary skill in the art.

### Formulation Example 1

### Pharmaceutical Composition Comprising Pancreatin

A pharmaceutical composition comprising: 10 kg of pancreatin is mixed with 2.5 kg of ethylene glycol 4000 and 1.5 kg of propan-2-ol to give a mixture which was then extruded in an extruding press. Pancreatin micropellets are prepared as disclosed in EP 0 583 726 and can be further packed into capsules or sachets.

### Formulation Example 2

### Pancreatin Micropellets Coated with a Gastric Acid Resistant Coating

The pancreatin micropellets obtained by the preceding example can be provided with a gastric acid resistant coating. For example, the pancreatin micropellets can be coated with gastric-juice-resistant film-forming agents such as, e.g., hydroxypropylmethylcellulose acetate succinate (HPMCAS), hydroxypropylmethylcellulose phthalate (HPMCP), cellulose acetate phthalate (CAP) or polyvinyl acetate phthalate (PVAP). Copolymers known as film-forming agents such as, for example, methacrylic acid/methyl methacrylate copolymers or methacrylic acid/ethyl acrylate copolymers, can also be used. The film-forming agents can be applied to the pancreatin micropellets using various film-coating apparatus, e.g. coaters, in the customary use forms, e.g. as organic solutions or organic or aqueous dispersions, optionally with addition of a conventional plasticizer. The resulting gastric acid-resistant film-coated pancreatin micropellets are distinguished by a high bulk density, for example in the range from 0.6 g/ml to 0.85 g/ml, which makes it possible to increase the filling weight per capsule and thus the active compound content of each capsule. Further experimental details on the process for preparing the gastric acid-resistant film-coated pancreatin micropellets are disclosed in EP 0 583 726.

Examples of pharmaceutically acceptable excipients include binding agents such as polyethylene glycol 1500, polyethylene glycol 2000, polyethylene glycol 3000, polyethylene glycol 4000, polyethylene glycol 6000, polyethylene glycol 8000, polyethylene glycol 10000, hydroxypropyl methylcellulose, polyoxyethylen, copolymers of polyoxyethylen-polyoxypropylen and mixtures of said organic polymers. The foregoing list of pharmaceutically acceptable binding agents is not meant to be exhaustive, but merely illustrative as a person of ordinary skill in the art would understand that many other pharmaceutically acceptable binding agents or combination of binding agents could also be used. Polyethylene glycol 4000 is the preferred pharmaceutically acceptable binding agent.

Examples of additional pharmaceutically acceptable excipients include gliding agents like magnesium stearate or calcium stearate, stearic acid, talcum and/or starch; fillers like calcium phosphate, corn starch, dextrans, dextrin, hydrated silicon dioxide, microcrystalline cellulose, kaolin, lactose, mannitol, polyvinyl pyrrolidone, precipitated calcium carbonate, sorbitol and/or talcum; disintegrating agents like Aerosil™ (silicic acid), alginic acid, amylose, calcium alginate, calcium carbonate, formaldehyde gelatin, pectic carbonate, sago starch, sodium bicarbonate and/or starch; and/or moisturizers like glycerol and/or starch. The foregoing list of pharmaceutically acceptable excipients is not meant to be exhaustive, but merely illustrative as a person or ordinary skill in the art would understand that many other pharmaceutically acceptable excipients or combination of excipients could also be used.

Methods of Treat Pancreatin Exocrine Insufficiency

Maldigestion in mammals such as humans is usually based on a deficiency of digestive enzymes, in particular on a deficiency of endogenous lipase, but also of protease and/or amylase. The cause of such a deficiency of digestive enzymes is frequently a hypofunction of the pancreas (e.g. pancreatic insufficiency, usually known as pancreatic exocrine insufficiency), the organ which produces the largest quantity of, and the most important, endogenous digestive enzymes. If the pancreatic insufficiency is pathological, it may be congenital or acquired. Acquired chronic pancreatic insufficiency may, for example, result from alcoholism. Congenital pancreatic insufficiency may, for example, result from disease such as cystic fibrosis. The consequences of the deficiency of digestive enzymes may be severe symptoms of under-nutrition and malnutrition, which may be accompanied by increased susceptibility to secondary illnesses. One embodiment is a method of treating pancreatin exocrine insufficiency of any origin in a mammalian subject comprising the step of administering the pharmaceutical compositions described herein.

In yet another embodiment, a method is provided for the treatment of a medical condition such as digestive disorders, pancreatic exocrine insufficiency, pancreatitis, cystic fibrosis, diabetes type I and/or diabetes type II by administering a therapeutically effective amount of a pharmaceutical composition described herein to a person in need of such treatment.

All references, including publications, patent applications, and patents, cited herein are hereby incorporated by reference to the same extent as if each reference were individually and specifically indicated to be incorporated by reference and were set forth in its entirety herein.

The use of the terms “a,” “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the present disclosure.

Alternative embodiments of the claimed disclosure are described herein, including the best mode known to the inventors for practicing the claimed invention. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

We claim:

1. A method for separating components of a pancreatin specimen comprising a viral load, the method comprising the steps of:
a) producing a liquid pancreatin test sample suitable for centrifugation from the pancreatin specimen without substantially altering the viral load thereof;
b) subjecting the pancreatin test sample from step a) to centrifugation wherein viruses with sedimentation constants greater than about 120 S do not form a pellet;
c) discarding any solid deposit optionally arising in process step b) during centrifugation and retaining the resulting test sample supernatant;
d) subjecting a portion of the pancreatin test sample supernatant obtained in step c) to a first ultracentrifugation in a discontinuous gradient medium comprising a lowest concentration gradient component and a next higher concentration gradient component, wherein the duration of the first ultracentrifugation and the relative centrifugal force for the first ultracentrifugation are selected such that a portion of the viral load is transported out of the pancreatin test sample supernatant into a first target fraction situated above or in the boundary layer between the lowest concentration gradient component and the next higher concentration gradient component, and removing the first target fraction which (i) contains a portion of the viral load from the pancreatin test sample supernatant; and (ii) may optionally include a pellet present after centrifugation;
e) subjecting the first target fraction obtained in step d) to a second ultracentrifugation wherein the second ultracentrifugation is carried out with a relative centrifugal force higher than the relative centrifugal force of the first ultracentrifugation on a gradient medium wherein the gradient medium is at a higher concentration than the lowest concentration gradient component of the first target fraction; and
f) obtaining a second target fraction containing a portion of the viral load by removing about the upper 75% (vol./vol.) of liquid of an upper layer which corresponds to the first target fraction and obtaining a lower fraction corresponding to (i) about the lower 25% (vol./vol.) liquid of the upper layer; and (ii) the complete layer of the gradient medium of step e) excluding any pellet optionally present after centrifugation.

2. The method of claim 1 further comprising quantitatively determining the virus infection titer in the second target fraction containing the viral load.

3. The method of claim 2, in which a diluted or an undiluted second target fraction is filtered through a microfilter before quantitatively determining the viral load.

4. The method of claim 1 wherein in step a) the pancreatin test sample is produced as a pancreatin test sample suspension by combining the pancreatin specimen with (i) a cell culture medium which is suitable for the cell line used to culture the virus type to be investigated; or (ii) a saline solution; and with one or more antibiotics.

5. The method of claim 4 wherein at least one of steps a) to f) are carried out with cooling to a temperature of between about 0° C. and about 15° C.

6. The method of claim 1 wherein centrifugation in process step b) is carried out at a relative centrifugal force of less than about 15,000×g.

7. The method of claim 1 wherein centrifugation in step b) is carried out at a relative centrifugal force of between about 8,000×g and about 15,000×g.

8. The method of claim 1 wherein centrifugation in step b) is carried out for a duration of greater than about five minutes.

9. The method of claim 1 wherein the first ultracentrifugation in step d) is carried out for duration of greater than about nine hours.

10. The method of claim 1 wherein the first ultracentrifugation in step d) is carried out at a relative centrifugal force of less than about 150,000×g.

11. The method of claim 1 wherein the second ultracentrifugation in step e) is carried out for a duration of greater than about two hours.

12. The method of claim 11 wherein the second ultracentrifugation in step e) is carried out at a relative centrifugal force of greater than about 150,000×g.

13. The method of claim 1 wherein the discontinuous gradient medium of step d) is a discontinuous two-phase sucrose gradient.

14. The method of claim 13 wherein the discontinuous gradient medium is a gradient comprising about a 50% (wt./vol.) buffered sucrose solution and about a 20% (wt./vol.) buffered sucrose solution.

15. The method of claim 1 wherein the higher concentration gradient medium in step e) is a gradient medium of about 50% (wt./vol.) buffered sucrose solution.

16. The method of claim 1 wherein the pancreatin specimen is a porcine-derived pancreatin specimen.

17. The method of claim 1 wherein the viral load of the pancreatin test sample comprises one or more viruses selected from the group consisting of: bovine rotavirus A, encephalomyocarditis virus, porcine circovirus, porcine parvovirus, porcine rotavirus A, porcine teschovirus, porcine hepatitis E virus and swine vesicular disease virus.

18. An isolated second target fraction, obtainable by the method of claim 1.

19. The isolated second target fraction as claimed in claim 19, wherein in step a) of claim 1 the pancreatin test sample is produced as a pancreatin test sample suspension by combining the pancreatin specimen with (i) a cell culture medium which is suitable for the cell line used to culture the virus type to be investigated; or (ii) a saline solution; and with one or more antibiotics.

20. The isolated second target fraction as claimed in claim 20, in which the saline solution is a phosphate buffered saline solution.

21. A method of treating pancreatin exocrine insufficiency in a mammalian subject comprising the steps of:
a) combining pancreatin having a viral load with one or more pharmaceutically acceptable excipients to create a dosage form suitable for oral administration;
b) separating the viral load of a pancreatin specimen derived from the pancreatin of step a) by the process comprising the steps of:
i) producing a liquid pancreatin test sample suitable for centrifugation from the pancreatin specimen without substantially altering the viral load thereof;
ii) subjecting the pancreatin test sample from step a) to centrifugation wherein viruses with sedimentation constants greater than about 120 S do not form a pellet;
iii) discarding any solid deposit optionally arising in process step b) during centrifugation and retaining the resulting test sample supernatant;
iv) subjecting a portion of the pancreatin test sample supernatant obtained in step c) to a first ultracentrifugation in a discontinuous gradient medium comprising a lowest concentration gradient component and a next higher concentration gradient component, wherein the duration of the first ultracentrifugation and the relative centrifugal force for the first ultracentrifugation are selected such that a portion of the viral load is transported out of the pancreatin test sample supernatant into a first target fraction situated above or in the boundary layer between the lowest concentration gradient component and the next higher concentration gradient component, and removing the first target fraction which (i) contains a portion of the viral load from the pancreatin test sample supernatant; and (ii) may optionally include a pellet present after centrifugation;
v) subjecting the first target fraction obtained in step d) to a second ultracentrifugation wherein the second ultracentrifugation is carried out with a relative centrifugal force higher than the relative centrifugal force of the first ultracentrifugation on a gradient medium wherein the gradient medium is at a higher concentration than the lowest concentration gradient component of the first target fraction; and
vi) obtaining a second target fraction containing a portion of the viral load by removing about the upper 75% (vol./vol.) of liquid of an upper layer which corresponds to the first target fraction and obtaining a lower fraction corresponding to (i) about the lower 25% (vol./vol.) liquid of the upper layer; and (ii) the complete layer of the gradient medium of step e) excluding any pellet optionally present after centrifugation; and
c) orally administering the dosage form to the subject in an amount sufficient to treat the pancreatic exocrine insufficiency.

22. A pharmaceutical composition comprising:
a) a pharmaceutically effective quantity of pancreatin in an oral dosage form, wherein the viral load of the pancreatin is quantitatively determined by the process comprising the steps of:
i) producing a liquid pancreatin test sample suitable for centrifugation from the pancreatin specimen without substantially altering the viral load thereof;
ii) subjecting the pancreatin test sample from step a) to centrifugation wherein viruses with sedimentation constants greater than about 120 S do not form a pellet;
iii) discarding any solid deposit optionally arising in process step b) during centrifugation and retaining the resulting test sample supernatant;
iv) subjecting a portion of the pancreatin test sample supernatant obtained in step c) to a first ultracentrifugation in a discontinuous gradient medium comprising a lowest concentration gradient component and a next higher concentration gradient component, wherein the duration of the first ultracentrifugation and the relative centrifugal force for the first ultracentrifugation are selected such that a portion of the viral load is transported out of the pancreatin test sample supernatant into a first target fraction situated above or in the boundary layer between the lowest concentration gradient component and the next higher concentration gradient component, and removing the first target fraction which (i) contains a portion of the viral load from the pancreatin test sample supernatant; and (ii) may optionally include a pellet present after centrifugation;
v) subjecting the first target fraction obtained in step d) to a second ultracentrifugation wherein the second ultracentrifugation is carried out with a relative centrifugal force higher than the relative centrifugal force of the first ultracentrifugation on a gradient medium of the same type as used in the first ultracentrifugation wherein the gradient medium is at a higher concentration than the lowest concentration gradient component of the first target fraction; and
vi) obtaining a second target fraction containing a portion of the viral load by removing about the upper 75% (vol./vol.) of liquid of an upper layer which corresponds to the first target fraction and obtaining a lower fraction corresponding to (i) about the lower 25% (vol./vol.) liquid of the upper layer; and (ii) the complete layer of the gradient medium of step e) excluding any pellet optionally present after centrifugation;
vii) determining the viral titer of the resulting viral load; and
b) one or more pharmaceutically acceptable excipients.

23. A pharmaceutical composition prepared by a process comprising the steps of:
a) providing quantity of pancreatin having a viral load, wherein the viral load of the pancreatin has been quantitatively determined by the process comprising the steps of:
i) producing a liquid pancreatin test sample suitable for centrifugation from the pancreatin specimen without substantially altering the viral load thereof;
ii) subjecting the pancreatin test sample from step a) to centrifugation wherein viruses with sedimentation constants greater than about 120 S do not form a pellet;
iii) discarding any solid deposit optionally arising in process step b) during centrifugation and retaining the resulting test sample supernatant;
iv) subjecting a portion of the pancreatin test sample supernatant obtained in step c) to a first ultracentrifugation in a discontinuous gradient medium comprising a lowest concentration gradient component and a next higher concentration gradient component, wherein the duration of the first ultracentrifugation and the relative centrifugal force for the first ultracentrifugation are selected such that a portion of the viral load is transported out of the pancreatin test sample supernatant into a first target fraction situated above or in the boundary layer between the lowest concentration gradient component and the next higher concentration gradient component, and removing the first target fraction which (i) contains a portion of the viral load from the pancreatin test sample supernatant; and (ii) may optionally include a pellet present after centrifugation;
v) subjecting the first target fraction obtained in step d) to a second ultracentrifugation wherein the second ultracentrifugation is carried out with a relative centrifugal force higher than the relative centrifugal force of the first ultracentrifugation on a gradient medium of the same type as used in the first ultracentrifugation wherein the gradient medium is at a higher concentration than the lowest concentration gradient component of the first target fraction; and
vi) obtaining a second target fraction containing a portion of the viral load by removing about the upper 75% (vol./vol.) of liquid of an upper layer which corresponds to the first target fraction and obtaining a lower fraction corresponding to (i) about the lower 25% (vol./vol.) liquid of the upper layer; and (ii) the complete layer of the gradient medium of step e) excluding any pellet optionally present after centrifugation;
vii) determining the viral titer of the resulting viral load; and
b) reducing the viral load of the pancreatin; and
c) combining the pancreatin having a reduced viral load with one or more pharmaceutically acceptable excipients and creating a dosage form suitable for oral administration.
